(19) United States
(12) Patent Application Publication
(10) Pub. No.: US 2015/0282759 A1
(43) Pub. Date: Oct. 8, 2015

(54) METHOD OF MEASURING SIGNALS FOR WEARABLE ELECTRONIC DEVICE AND WEARABLE ELECTRONIC DEVICE USING THE SAME

(71) Applicant: Wistron Corporation, New Taipei City (TW)

(72) Inventors: Pai-Yang Lin, New Taipei City (TW); HONG-CHI CHEN, New Taipei City (TW)

(21) Appl. No.: 14/470,918

(22) Filed: Aug. 27, 2014

(30) Foreign Application Priority Data
Apr. 7, 2014 (TW) 103112731

(51) Int. Cl. A61B 5/00; A61B 5/0476; A61B 5/01; A61B 5/024; A61B 5/08

(52) U.S. Cl. CPC A61B 5/6802; A61B 5/02405; A61B 5/0816; A61B 5/01; A61B 5/0476

(57) ABSTRACT

A method of measuring signals for a wearable electronic device includes setting at least one threshold value corresponding to each of a plurality of physiological signals of a user; measuring the plurality of physiological signals to obtain a plurality of measurement data; and obtaining a measurement result according to the threshold value and the plurality of measurement data.

### BACKGROUND OF THE INVENTION

1. Field of the Invention

The present invention relates to a method of measuring signals and a related wearable electronic device, and more particularly, to a method of measuring signals capable of measuring various human physiological signals and a related wearable electronic device.

2. Description of the Prior Art

With development of wearable technology, products such as wearable cameras, smart watches and Google Glasses are expected to be the mainstream of future consumer electronic products. The wearable products can be worn on the body of a user, which facilitates the measurement of human physiological information; hence, wearable products can guide development in the fields of health care and sport via the combination of measurement devices for obtaining different kinds of physiological information.

Commercially available wearable devices have a measurement function for only one type of physiological information. For example, an earphone may be equipped with a temperature sensor for measuring ear temperature of the user. A watch may be equipped with a heart rate sensor to measure the heart rate of the user by sensing his/her pulse. These devices, however, cannot simultaneously perform measurement of different types of physiological signals, and thus cannot combine measurement data of different types of physiological signals or human actions to perform subsequent analysis. When a user needs to obtain many types of physiological information for sport or health care reasons, the user needs to wear many wearable devices at the same time for obtaining all measurement data. In addition, the information obtained by the abovementioned devices should first be transmitted to a processing device, such as a computer or smart phone, for data analysis and processing. Variations in the physiological information cannot be obtained immediately, which is inconvenient. Furthermore, although current wearable devices detect and record the physiological signals at all times, they lack definite time points for detection and timely feedback mechanisms. Thus, there is room for improvement over the prior art.

### SUMMARY OF THE INVENTION

It is therefore an objective of the present invention to provide a method of measuring signals and a related wearable electronic device capable of measuring various human physiological signals, action signals and location information, and capable of providing timely information for a user according to the measured signals and location information.

The present invention discloses a method of measuring signals for a wearable electronic device. The method comprises setting at least one threshold value corresponding to each of a plurality of physiological signals of a user; measuring the plurality of physiological signals to obtain a plurality of measurement data; and obtaining a measurement result according to the threshold value and the plurality of measurement data.

The present invention further discloses a method of measuring signals for a wearable electronic device. The method comprises setting at least one threshold value corresponding to each of at least one physiological signal of a user; detecting a status of the user to generate a status signal; measuring the physiological signal and obtaining at least one measurement data according to a relationship between the status signal and the physiological signal; and obtaining a measurement result according to the threshold value, the status signal and the measurement data.

The present invention further discloses a wearable electronic device. The wearable electronic device comprises a threshold value setting device for setting at least one threshold value corresponding to each of at least one physiological signal; at least one physiological signal measurement device, for measuring a corresponding physiological signal to obtain at least one measurement data; and a physiological signal determination module, for obtaining a measurement result according to the threshold value and the measurement data.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of the structure of a wearable electronic device according to an embodiment of the present invention.

FIG. 2A is a schematic diagram of a process according to an embodiment of the present invention.

FIG. 2B is a schematic diagram of the measurement data corresponding to the units of time according to an embodiment of the present invention.

FIG. 3A is a schematic diagram of a process according to an embodiment of the present invention.

FIG. 3B is a schematic diagram of a time interval between an occurrence of the physiological signal and an occurrence of the next or later physiological signal according to an embodiment of the present invention.

FIG. 4A is a schematic diagram of a process according to an embodiment of the present invention.

FIG. 4B is a schematic diagram of the measurement data corresponding to the units of moving distance of the user according to an embodiment of the present invention.

FIG. 5A is a schematic diagram of a process according to an embodiment of the present invention.

FIG. 5B is a schematic diagram of the moving distance of the user during a time interval between an occurrence of the physiological signal and an occurrence of the previous or former physiological signal according to an embodiment of the present invention.

FIG. 6A is a schematic diagram of a process according to an embodiment of the present invention.

FIG. 6B is a schematic diagram of the number of times an action is executed by the user during a time interval between an occurrence of the physiological signal and an occurrence of the next or later physiological signal according to an embodiment of the present invention.

FIG. 7A is a schematic diagram of a process according to an embodiment of the present invention.

FIG. 7B is a schematic diagram of the measurement data corresponding to the unit of actions executed by the user according to an embodiment of the present invention.

### DETAILED DESCRIPTION

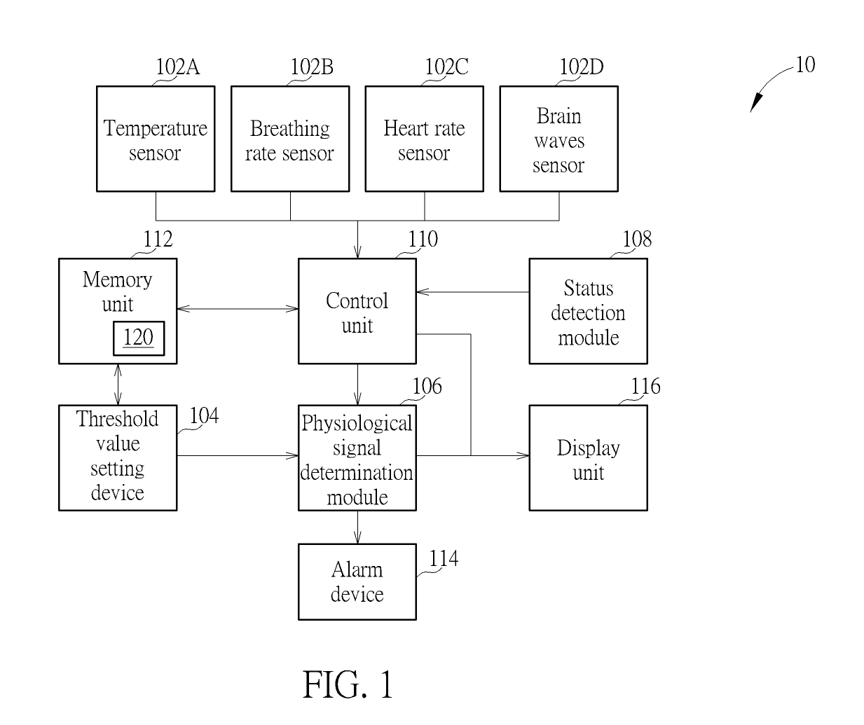

Please refer to FIG. 1, which is a schematic diagram of the structure of a wearable electronic device 10 according to an embodiment of the present invention. As shown in FIG. 1, the wearable electronic device 10 includes physiological signal measurement devices 102A-102D, a threshold value setting device 104, a physiological signal determination module 106, a status detection module 108, a control unit 110, a memory unit 112, an alarm device 114 and a display unit 116. The wearable electronic device 10 is utilized for measuring various physiological signals such as body temperature, breathing rate, heart rate and brain waves. The physiological signal measurement devices 102A-102D may be various measurement devices capable of measuring the physiological signals. Such devices may be utilized for measuring various physiological signals to obtain measurement data. In this embodiment, the physiological signal measurement devices 102A-102D include a temperature sensor 102A, a breathing rate sensor 102B, a heart rate sensor 102C and a brain waves sensor 102D, which may be utilized for measuring the body temperature, breathing rate, heart rate and brain waves, respectively. Please note that, in other embodiments, the wearable electronic device 10 may also include or be externally connected to other types of physiological signal measurement devices such as an electrocardiography device, sphygmomanometer and oximeter, in order to perform measurement on other physiological signals according to user requirements.

For the physiological signal measurement functionalities of the physiological signal measurement devices 102A-102D, the threshold value setting device 104 may set a corresponding threshold value or reference data, e.g. an upper or lower limit of the body temperature, a normal range of the heart rate or breathing rate, etc. These threshold values or reference data may be set by the user, or may be determined according to standard values provided by the World Health Organization (WHO), medical academies or sports science research institutions. The threshold value setting device 104 may also determine the corresponding threshold values or reference data for the detected status parameters such as time, relative distance and altitude.

The physiological signal determination module 106 may obtain a measurement result according to the threshold value predefined by the threshold value setting device 104 and the measurement data obtained by the physiological signal measurement devices 102A-102D. The physiological signal determination module 106 may obtain different measurement results corresponding to different measurement purposes or different physiological signals. The physiological signal determination module 106 may also be utilized for triggering a time point of the measurement, and performing determination on the data calculated according to subsequent steps. For example, the physiological signal determination module 106 may compare the threshold value with the measurement data obtained by the physiological signal measurement devices 102A-102D, and may learn whether a physiological signal of the user is within a normal range, in order to determine the physiology of the user, e.g. loss of temperature or under an attack of fever, normal or abnormal heart rate, mentally fit or unfit, etc.

The status detection module 108 may be utilized for detecting the status of a user to generate a status signal. The status of the user may include the user's location, altitude, action or motion. If the user uses the wearable electronic device 10 when exercising or climbing mountains, the status detection module 108 may obtain the status signal according to the user's action, motion or location, in order to combine the status signal with the measurement data of the physiological signals obtained by the physiological signal measurement devices 102A-102D, which provides proper information for the user.

The user may use the wearable electronic device 10 for marathon training, and the status detection module 108 may utilize a global positioning system (GPS) for determining the location of the user, in order to calculate the running distance of the user. The wearable electronic device 10 may also divide the 42-kilometer distance of the marathon into two 21-kilometer sections, and the physiological signal measurement devices 102A-102D may obtain the physiological signals (e.g. the number of breathing and heartbeat) of the user in the first-half and the second-half, respectively. The user may perform pace adjustment according to the difference in breathing rates or heart rates between the first-half and the second-half. For example, in the first-half, the running records of the athlete may be applied to for reference and incorporated with the measurement data of physiological signals allowing the wearable electronic device 10 to perform determination, which provides timely feedback signals to the athlete for breathing adjustment in order to maintain their physical strength. In the second-half, the wearable electronic device 10 may provide pacing or accelerating indications for the athlete according to a predefined threshold value. The status detection module 108 may include various detectors such as a GPS, a gravity sensor (G-sensor), a gyroscope or an atmospheric pressure detector, in order to detect various status information of the user.

The control unit 110 may be utilized for controlling entire operations of the wearable electronic device 10. As shown in FIG. 1, the control unit 110 receives the measurement data of physiological signals from the physiological signal measurement devices 102A-102D and the status signals from the status detection module 108, and controls the physiological signal determination module 106 to obtain the measurement results according to user requirements. The control unit 110 may include a microcontroller or microprocessor for executing the operations and computations for the various physiological signals, e.g. calculating the variations in measurement data of physiological signals or the average value during a unit of time, etc. The conversion of various signals may also be performed by the control unit 110.

The memory unit 112 may be utilized for storing the abovementioned measurement data and measurement results. In addition, the memory unit 112 stores the program code 120 for instructing the microcontroller or microprocessor of the control unit 110 to execute the signal processing and operations. The memory unit 112 may be any type of memory device such as a random access memory (RAM), a read-only memory (ROM), a flash memory or other memory devices. The alarm device 114 may generate a warning signal according to the measurement result obtained from the physiological signal determination module 106. The warning signal may be delivered through vibration, sound, light or any other form. The alarm device 114 may deliver the warning signal in a proper form according to the characteristics of the wearable electronic device 10. The display unit 116 may be utilized for displaying the abovementioned measurement data, measurement results or warning signals. For example, the display unit 116 may include a small screen disposed on a watch or glasses to display the measurement data or the measurement results.

Preferably, the display unit 116 may display the measurement data of physiological signals or the measurement results of the user. In comparison with the conventional wearable devices that have to transmit the measurement data or measurement results to a mobile phone or computer for displaying, the present invention may provide the physiological information for the user in real time. The user may not need to carry a mobile phone when exercising, and the physiological information may be obtained only from the information timely displayed by the display unit 116 or the warning signals provided by the alarm device 114, so that the user can take steps accordingly.

Through the abovementioned structure of the wearable electronic device 10, the measurement of various physiological signals can be realized. In an embodiment, the concentration of a user may be determined by measuring the brain waves and breathing rate of the user. When the user is in a high concentration mood with no distraction, alpha waves of the brain waves having a frequency around 11-12 Hertz (Hz) may be detected. In addition, when a person is motionless, the breathing rate of the person may be under 10-18 times per minute. Thus, when the user performs an activity requiring high concentration (e.g. archery), the breathing rate sensor 102B and the brain wave sensor 102D may be utilized for measuring the user's breathing rate and brain waves, respectively. The threshold value setting device 104 may set a lower threshold value and an upper threshold value of the breathing rate to 10 times per minute and 18 times per minute, respectively, and set a lower threshold value and an upper threshold value of the frequency of alpha waves to 11 Hz and 12 Hz, respectively. The alarm device 114 may send a warning signal when the physiological signal determination module 106 determines that the user's breathing rate and brain waves are both located within the range of the threshold values, in order to indicate the user is in a high concentration mood.

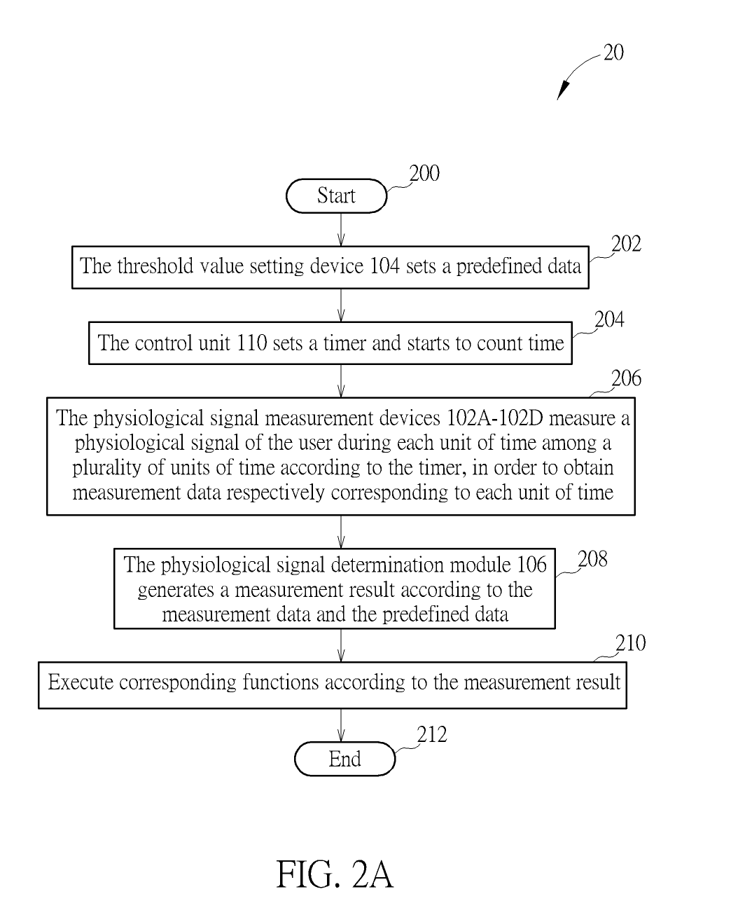

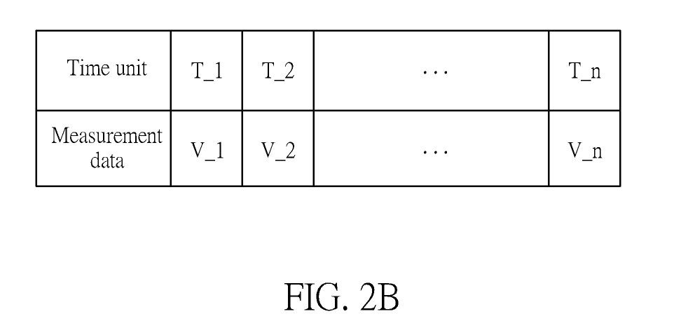

Please refer to FIG. 2A, which is a schematic diagram of a process 20 according to an embodiment of the present invention. As shown in FIG. 2A, the process 20 may be utilized in the wearable electronic device 10 for detecting the physiological signals of the user during each unit of time, in order to obtain measurement data corresponding to each unit of time. The process 20 includes the following steps:

Step 200: Start.

Step 202: The threshold value setting device 104 sets a predefined data.

Step 204: The control unit 110 sets a timer and starts to count time.

Step 206: The physiological signal measurement devices 102A-102D measure a physiological signal of the user during each unit of time among a plurality of units of time according to the timer, in order to obtain measurement data respectively corresponding to each unit of time.

Step 208: The physiological signal determination module 106 generates a measurement result according to the measurement data and the predefined data.

Step 210: Execute corresponding functions according to the measurement result.

Step 212: End.

In the process 20, the threshold value setting device 104 first sets the predefined data corresponding to the physiological signal. For example, if the user needs to measure whether he/she can maintain concentration when playing chess, the threshold value setting device 104 may predefine a threshold value (i.e. 90%) and determine whether the ratio of alpha waves to total brain waves in each unit of time reaches 90%. The predefined data may be manually inputted by the user or provided from the standard values of official institutions, and may be adjusted personally according to the formerly measured physiological information of the user. The control unit 110 then sets a timer, and starts to count time when the user needs to perform measurement of the physiological signals.

As shown in FIG. 2B, the physiological signal measurement devices 102A-102D may measure the physiological signal of the user during a plurality of time units T₁1, T_2, . . . , T_n according to the timer, in order to obtain measurement data V_1, V_2, . . . , V_n corresponding to each of the plurality of time units T_1, T_2, . . . , T_n, respectively. For example, after the timer starts to count time, the user may set a time unit to 10 minutes, and the brain wave sensor 102D may detect the distribution of the brain waves of the user in each 10 minute interval. The physiological signal determination module 106 then generates the measurement result according to the measurement data V_1-V_n and the predefined data. The control unit 110, the alarm device 114 or the display unit 116 may further execute corresponding functions according to the measurement result. In the above embodiment, if the ratio of the alpha waves measured during a unit of time does not achieve 90%, the physiological signal determination module 106 may determine that the user's concentration is not high enough. The alarm device 114 thereby delivers a warning signal, which may be delivered through vibrations, sounds, etc. The display unit 116 may also display information indicating that the user's concentration is not high enough. If the ratio of the alpha waves measured during a unit of time achieves 90%, the physiological signal determination module 106 may determine that the user's concentration meets a certain requirement. The control unit 110 or the alarm device 114 may not execute any functions, or the display unit 116 may display information indicating that the user's concentration meets the requirement. Please note that the functions executed by the control unit 110 according to the above measurement result may include any corresponding data processing or signal transmission.

The step of the physiological signal determination module 106 generating the measurement result according to the measurement data V_1-V_n and the predefined data is not only limited to the comparison between the measurement data V_1-V_n and the predefined data (e.g. comparing whether the ratio of the alpha waves occurring in a unit of time reaches 90%). For example, the measurement data V_1-V_n measured during a unit of time may undergo further operations or computations, e.g. determining the trend of data variation (increasing or decreasing), estimating measurement data in a specific time in the future, or calculating the average of the measurement data V_1-V_n, etc. The measurement data of different types of physiological signals obtained in each unit of time may also be utilized for estimating other types of data. In an embodiment, the alarm device 114 may deliver a warning signal when the measured ratio of alpha waves decreases progressively in three consecutive time units, in order to indicate that the user's concentration is falling. The alarm device 114 or the control unit 110 may also execute corresponding functions according to other computational results of the measurement data V_1-V_n; this is not limited herein.

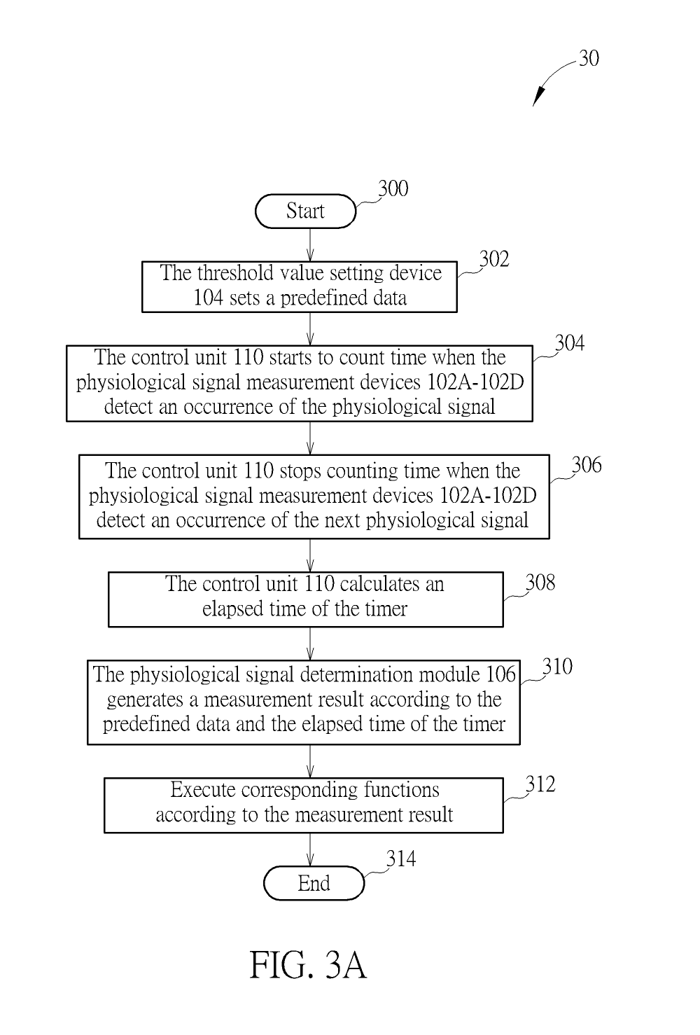

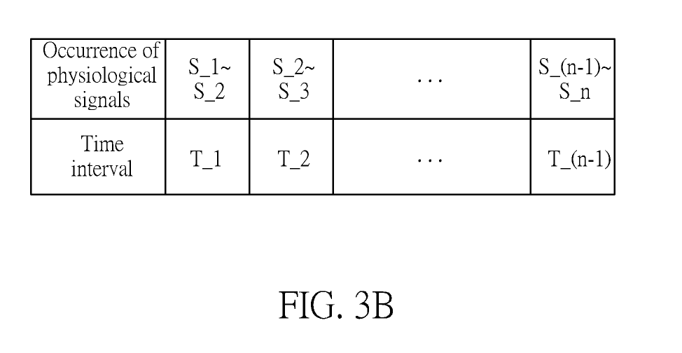

Please refer to FIG. 3A, which is a schematic diagram of a process 30 according to an embodiment of the present invention. As shown in FIG. 3A, the process 30 may be utilized in the wearable electronic device 10 for determining a time interval between an occurrence of the physiological signal and an occurrence of the next or later physiological signal. The process 30 includes the following steps:

Step 300: Start.

Step 302: The threshold value setting device 104 sets a predefined data.

Step 304: The control unit 110 starts to count time when the physiological signal measurement devices 102A-102D detect an occurrence of the physiological signal.

Step 306: The control unit 110 stops counting time when the physiological signal measurement devices 102A-102D detect an occurrence of the next physiological signal.

Step 308: The control unit 110 calculates an elapsed time of the timer.

Step 310: The physiological signal determination module 106 generates a measurement result according to the predefined data and the elapsed time of the timer.

Step 312: Execute corresponding functions according to the measurement result.

Step 314: End.

In the process 30, the threshold value setting device 104 first sets the predefined data corresponding to the physiological signal. For example, the user may need to measure a time interval between each breath, in order to determine whether the breathing rate is steady when doing long distance running. The threshold value setting device 104 may predefine a criterion where the time interval between each breath should be greater than 1 second. If the time interval is smaller than 1 second, the user may be determined to be out of breath.

The user then starts the training. As shown in FIG. 3B, the control unit 110 may start to count time when the physiological signal measurement devices 102A-102D detect the occurrence of a physiological signal S_1, and stop counting time when the physiological signal measurement devices 102A-102D detect the occurrence of the next physiological signal S_2, in order to calculate an elapsed time of the timer T_1. For example, the control unit 110 may start to count time at a time point when the breathing rate sensor 102B detects that the user starts to inhale, and stop counting time at a time point when the next inhalation of the user is detected, in order to calculate the time interval between these two breaths. Please note that Steps 304 to 308 may be executed repeatedly; hence, the physiological signal measurement devices 102A-102D may obtain time intervals T_1-T_(n−1) during the occurrence of physiological signals S_1-S_n, respectively. The physiological signal determination module 106 then generates the measurement result according to the predefined data and the elapsed time of the timer. The control unit 110, the alarm device 114 or the display unit 116 may further execute corresponding functions according to the measurement result. In the above embodiment, if a time interval T_x is smaller than 1 second, the physiological signal determination module 106 may determine that the user is out of breath, and the alarm device 114 may deliver a warning signal, where the warning signal may be vibration, sounds, etc. The display unit 116 may also display information indicating that the user is out of breath. If a time interval T_y is greater than 1 second, the user's breathing may be determined to be steady. The control unit 110 or the alarm device 114 may not execute any functions, or the display unit 116 may display the information indicating that the user's breathing is steady.

The step of the physiological signal determination module 106 generating the measurement result according to the predefined data and the elapsed time of the timer is not only limited to the comparison of the time intervals T_1-T_(n−1) and the predefined data (e.g. comparing whether each time interval is greater than 1 second). For example, the time intervals T_1-T_(n−1) measured according to the occurrence of each physiological signals S_1-S_n may undergo further operations or computations, e.g. converting to an accumulated time interval corresponding to the occurrence of multiple physiological signals (such as the time interval between the first breath and the tenth breath), determining the trend of data variation (increasing or decreasing), estimating the number of occurrences of the physiological signal during a specific unit of time in the future, or calculating the average of the time intervals T_1-T_(n−1), etc. In an embodiment, the alarm device 114 may also deliver a warning signal when the physiological signal determination module 106 determines that two consecutive time intervals T_z and T_(z+1) have a larger difference, in order to indicate that the user is out of breath. The alarm device 114 or the control unit 110 may also execute corresponding functions according to other computational results of the time intervals T_1-T_(n−1); this is not limited herein.

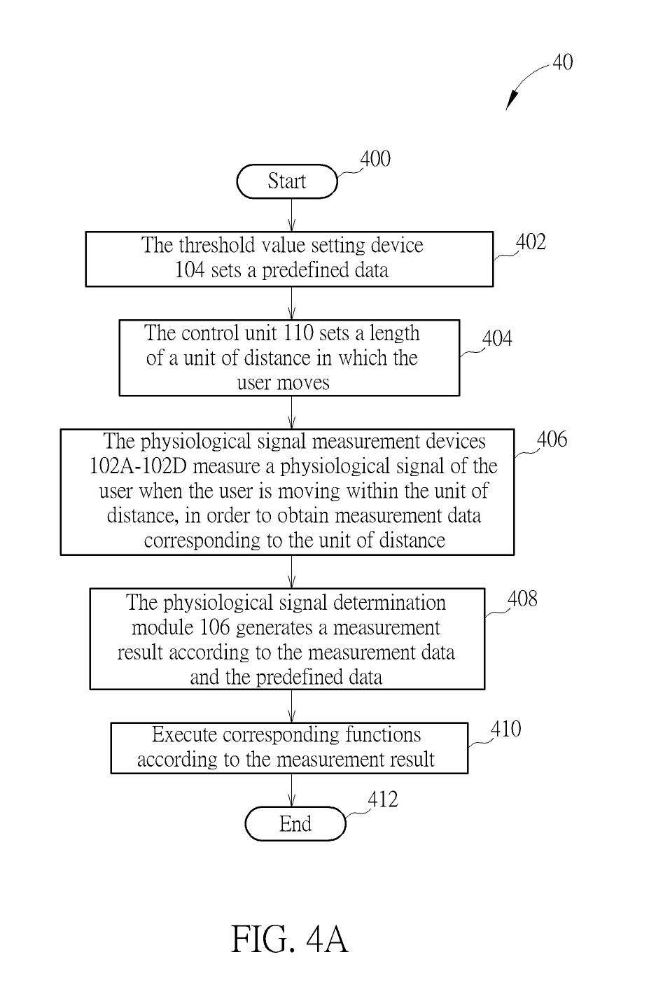

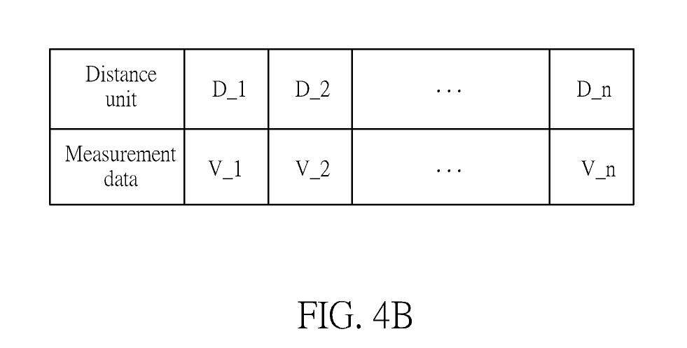

Please note that if the measurement data of the physiological signals is further combined with status information of the user, various applications may be realized. Please refer to FIG. 4A, which is a schematic diagram of a process 40 according to an embodiment of the present invention. As shown in FIG. 4A, the process 40 may be utilized in the wearable electronic device 10 for measuring the physiological signals of the user when the user is moving within a unit of distance, in order to obtain the measurement data corresponding to each unit of distance. The process 40 includes the following steps:

Step 400: Start.

Step 402: The threshold value setting device 104 sets a predefined data.

Step 404: The control unit 110 sets a length of a unit of distance in which the user moves.

Step 406: The physiological signal measurement devices 102A-102D measure a physiological signal of the user when the user is moving within the unit of distance, in order to obtain measurement data corresponding to the unit of distance.

Step 408: The physiological signal determination module 106 generates a measurement result according to the measurement data and the predefined data.

Step 410: Execute corresponding functions according to the measurement result.

Step 412: End.

In the process 40, the threshold value setting device 104 first sets the predefined data corresponding to the physiological signal. The threshold value setting device 104 also predefines a unit of distance in which the user moves. For example, if the user needs to determine whether his/her breathing rate is steady during a training process of 200 meters when running a 200-meter race, the threshold value setting device 104 may predefine a length of a distance unit to 50 meters and determine whether the frequency of breaths measured in each distance unit is between 3-5. If the number of breaths measured in a 50-meter distance is greater than 5 or smaller than 3, the user may have poor regulation of respiration.

The user then starts to perform the training. As shown in FIG. 4B, the physiological signal measurement devices 102A-102D measure the physiological signal of the user when the user is moving within distance units D_1, D_2, . . . , D_n, in order to obtain measurement data V_1, V_2, . . . , V_n corresponding to each distance unit D_1, D_2, . . . , D_n. For example, after the user starts to run, the breathing rate sensor 102B may detect the number of breaths in the first 50 meters, the 50^{th}-100^{th} meters, the 100^{th}-150^{th} meters and the 150^{th}-200^{th} meters, respectively. The physiological signal determination module 106 then generates the measurement result according to the measurement data V_1-V_n and the predefined data. The control unit 110, the alarm device 114 or the display unit 116 may further execute corresponding functions according to the measurement result. In the above embodiment, if the physiological signal determination module 106 determines that the number of breaths measured within a unit of distance is greater than 5 or smaller than 3, the alarm device 114 may deliver a warning signal indicating that the regulation of respiration is poor, where the warning signal may be vibrations, sounds, etc. The display unit 116 may also display information indicating that the respiration regulation of the user is poor. If the number of breaths measured within a unit of distance is between 3-5, the physiological signal determination module 106 may determine that the user's respiration regulation meets a requirement. The control unit 110 or the alarm device 114 may not execute any functions, or the display unit 116 may display information indicating that the user's respiration regulation meets the requirement.

The step of the physiological signal determination module 106 generating the measurement result according to the measurement data V_1-V_n and the predefined data is not only limited to the comparison between the measurement data V_1-V_n and the predefined data (e.g. comparing whether the number of breaths measured within a unit of distance is between 3-5). For example, the measurement data V_1-V_n measured within the unit of distance may undergo further operations or computations, e.g. determining the trend of data variation (increasing or decreasing), estimating measurement data corresponding to a unit of distance in the future, or calculating the average of the data V_1-V_n, etc. The measurement data of different types of physiological signals obtained within each unit of distance may also be utilized for estimating other types of data. In an embodiment, the alarm device 114 may deliver a warning signal when the number of breaths measured within a plurality of consecutive distance units obviously increases or decreases, in order to indicate that the user's respiration regulation is poor. The alarm device 114 or the control unit 110 may also execute corresponding functions according to other computational results of the measurement data V_1-V_n; this is not limited herein.

Please note that, in this embodiment, the status detection module 108 may include a GPS or other devices or technologies capable of detecting the moving distance, in order to detect the location of the user. This allows the control unit 110 to determine the moving distance of the user according to the user's locations detected at different time points, which may further be utilized for determining the measurement data V_1-V_n of the user in each of the distance unit D_1-D_n.

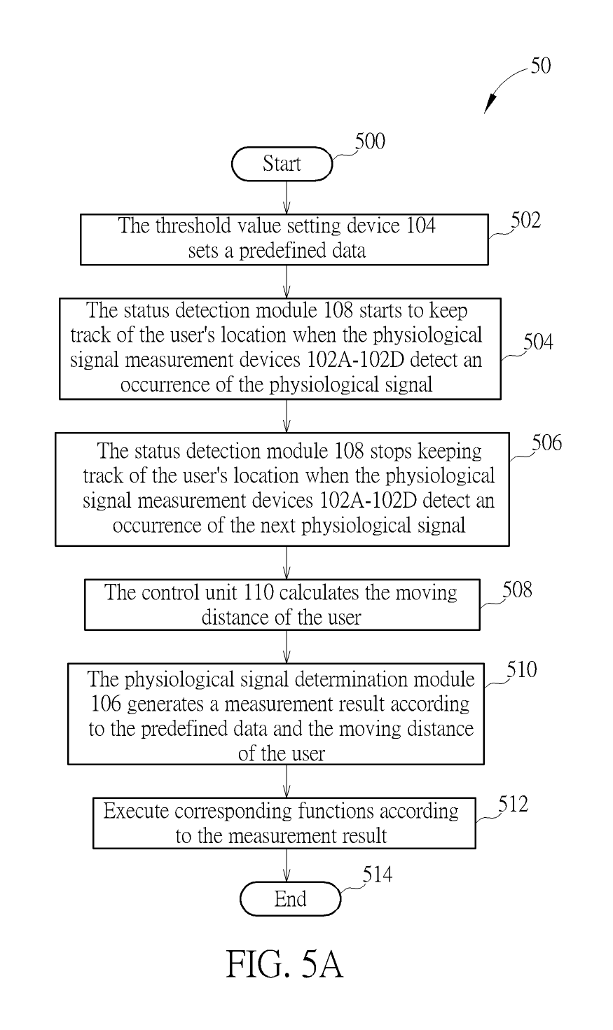

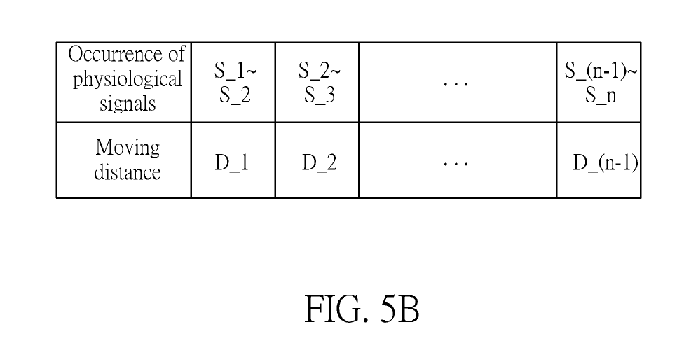

Please refer to FIG. 5A, which is a schematic diagram of a process 50 according to an embodiment of the present invention. As shown in FIG. 5A, the process 50 may be utilized in the wearable electronic device 10 for determining a moving distance of the user during a time interval between an occurrence of the physiological signal and an occurrence of the previous or former physiological signal. The process 50 includes the following steps:

Step 500: Start.

Step 502: The threshold value setting device 104 sets a predefined data.

Step 504: The status detection module 108 starts to keep track of the user's location when the physiological signal measurement devices 102A-102D detect an occurrence of the physiological signal.

Step 506: The status detection module 108 stops keeping track of the user's location when the physiological signal measurement devices 102A-102D detect an occurrence of the next physiological signal.

Step 508: The control unit 110 calculates the moving distance of the user.

Step 510: The physiological signal determination module 106 generates a measurement result according to the predefined data and the moving distance of the user.

Step 512: Execute corresponding functions according to the measurement result.

Step 514: End.

In the process 50, the threshold value setting device 104 first sets the predefined data corresponding to the physiological signal. For example, the user may need to measure the moving distance corresponding to each breath when doing long distance running, in order to determine whether the breathing rate is steady. The threshold value setting device 104 may predefine a criterion where the moving distance corresponding to each breath should be greater than 10 meters. If the corresponding moving distance is smaller than 10 meters, the user may be out of breath.

The user then starts the training. As shown in FIG. 5B, the status detection module 108 may start to keep track of the user's location when the physiological signal measurement devices 102A-102D detect the occurrence of a physiological signal S_1, and stops keeping track of the user's location when the physiological signal measurement devices 102A-102D detect the occurrence of the next physiological signal S_2, so that the control unit 110 may calculate the moving distance D_1 of the user accordingly. For example, the status detection module 108 may start to keep track of the user's location at a time point when the breathing rate sensor 102B detects that the user starts to inhale, and stop keeping track of the user's location at a time point when the next inhalation of the user is detected, in order to calculate the moving distance of the user between these two breaths. Please note that Steps 504 to 508 may be executed repeatedly; hence, the physiological signal measurement devices 102A-102D may obtain moving distances D_1-D_(n−1) during the occurrence of physiological signals S_1-S_n, respectively. The physiological signal determination module 106 then generates the measurement result according to the predefined data and the moving distance of the user. The control unit 110, the alarm device 114 or the display unit 116 may further execute corresponding functions according to the measurement result. In the above embodiment, if a moving distance D_x is less than 10 meters, the physiological signal determination module 106 may determine that the user breathes too early, and the alarm device 114 may deliver a warning signal indicating that the user breathes too early, where the warning signal may be vibration, sounds, etc. The display unit 116 may also display information indicating that the user breathes too early. If a moving distance D_y is greater than 10 meters, the user's breath may be steady. The control unit 110 or the alarm device 114 may not execute any functions, or the display unit 116 may display information indicating that the user's breath is steady enough.

The step of the physiological signal determination module 106 generating the measurement result according to the predefined data and the moving distances D_1-D_(n−1) of the user is not only limited to the comparison of the moving distances D_1-D_(n−1) and the predefined data (e.g. comparing whether each moving distance is greater than 10 meters). For example, the moving distances D_1-D_(n−1) measured according to the occurrence time of each of the physiological signals S_1-S_n may undergo further operations or computations, e.g. converting to an accumulated moving distance corresponding to the occurrence of multiple physiological signals (such as the moving distance between the first breath and the tenth breath), determining the trend of data variation (increasing or decreasing), estimating the number of occurrences of the physiological signal within a specific unit of distance, or calculating the average of the moving distances D_1-D_(n−1), etc. In an embodiment, the alarm device 114 may also deliver a warning signal when the physiological signal determination module 106 determines that two consecutive moving distances D_z and D_(z+1) have a larger difference, in order to indicate that the user is out of breath. The alarm device 114 or the control unit 110 may also execute corresponding functions according to other computational results of the moving distances D_1-D_(n−1); this is not limited herein.

Similarly, in this embodiment, the status detection module 108 may include a GPS or other related devices and technologies capable of detecting the user's location, in order to determine the moving distances D_1-D_(n−1) of the user between time points of two physiological signals according to the user's location at the time point of each physiological signal.

Please note that the method of measuring signals and the wearable electronic device of the present invention may measure various types of human physiological signals, action signals and location information, and provide information for the user according to the measured signals. Those skilled in the art can make modifications and variations accordingly. For example, in the embodiments shown in the processes 30 and 40, the status signal is the location and moving distance of the user and the GPS system is utilized for realizing the detection of the user's location. In other embodiments, the status signal may also include other statuses of the user such as the altitude or action. For example, the wearable electronic device 10 may measure the physiological signals when the user is at different altitudes, in order to obtain measurement data corresponding to different altitudes. In detail, the status detection module 108 may include a pressure sensor or other related devices utilized for detecting the altitude of the user. The physiological signal measurement devices 102A-102D may measure the physiological signals (e.g. body temperature, breathing rate or heart rate) of the user when the user is located at a first altitude and a second altitude, respectively, in order to obtain the measurement data corresponding to the first altitude and the second altitude. The control unit 110 then calculates the variations in measurement data corresponding to the altitude variations between the first altitude and the second altitude, in order to perform subsequent analysis and processing. This embodiment can determine whether the user is maladjusted due to loss of temperature, altitude sickness or other conditions when the user is climbing a mountain, in order to enable contingency plans. In another embodiment, the wearable electronic device 10 may use both the timer and the pressure sensor to detect the variations in altitude and the variations of physiological signals in each unit of time. The related implementations are similar to the abovementioned processes, and will not be narrated herein.

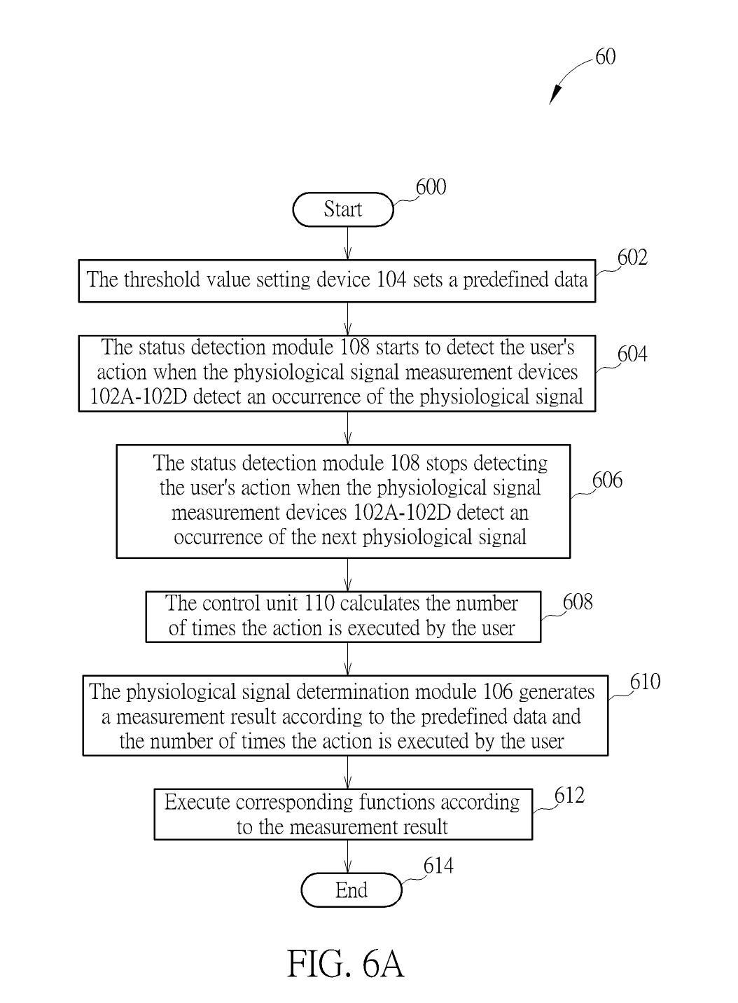

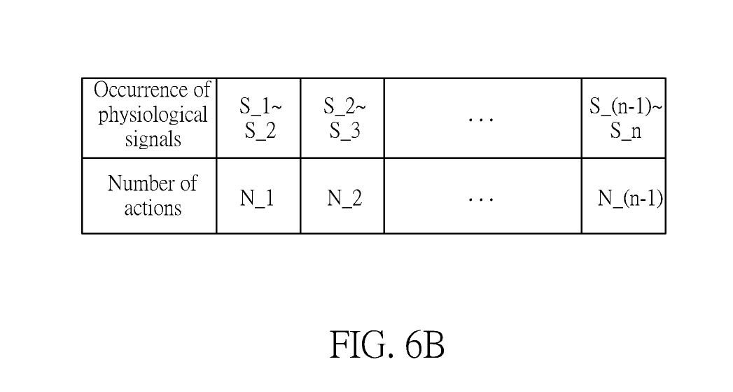

Please refer to FIG. 6A, which is a schematic diagram of a process 60 according to an embodiment of the present invention. As shown in FIG. 6A, the process 60 may be utilized in the wearable electronic device 10 for determining a number of times an action is executed by the user during a time interval between an occurrence of the physiological signal and an occurrence of the next or later physiological signal. The process 60 includes the following steps:

Step 600: Start.

Step 602: The threshold value setting device 104 sets a predefined data.

Step 604: The status detection module 108 starts to detect the user's action when the physiological signal measurement devices 102A-102D detect an occurrence of the physiological signal.

Step 606: The status detection module 108 stops detecting the user's action when the physiological signal measurement devices 102A-102D detect an occurrence of the next physiological signal.

Step 608: The control unit 110 calculates the number of times the action is executed by the user.

Step 610: The physiological signal determination module 106 generates a measurement result according to the predefined data and the number of times the action is executed by the user.

Step 612: Execute corresponding functions according to the measurement result.

Step 614: End.

In the process 60, the threshold value setting device 104 first sets the predefined data corresponding to the physiological signal. For example, the user may need to measure the number of reps of a dumbbell raise corresponding to each breath when performing weight training, in order to determine whether the breathing rate is steady as well as whether the raising speed is stable. The threshold value setting device 104 may predefine a criterion where the number of dumbbell reps corresponding to each breath (e.g. an action includes raising and lowering the dumbbell) should be performed between 2-3 times. If the action is performed less than 2 times, the user may be out of breath or the user's action may be too slow. If the action is performed more than 3 times, the user's breath may be too slow or the user's action may be too fast.

The user then starts the training. As shown in FIG. 6B, the status detection module 108 may start to detect the user's action when the physiological signal measurement devices 102A-102D detect the occurrence of a physiological signal S_1, and stop detecting the user's action when the physiological signal measurement devices 102A-102D detect the occurrence of the next physiological signal S_2, so that the control unit 110 may calculate the number of actions N_1 executed by the user accordingly. For example, the status detection module 108 may start to detect the user's action at a time point when the breathing rate sensor 102B detects that the user starts to inhale (inhalation is taken as an example herein; exhalation may also be applied for the detection time point), and stop detecting the user's action at a time point when the next inhalation of the user is detected, in order to calculate the number of actions executed by the user between these two breaths. Please note that Steps 604 to 608 may be executed repeatedly; hence, the physiological signal measurement devices 102A-102D may obtain the numbers of actions N_1-N_(n−1) during the occurrence of physiological signals S_1-S_n, respectively. The physiological signal determination module 106 then generates the measurement result according to the predefined data and the numbers of actions N_1-N_(n−1) executed by the user. The physiological signal determination module 106, the control unit 110, the alarm device 114 or the display unit 116 may further execute corresponding functions according to the measurement result. In the above embodiment, if a number of actions N_x is smaller than 2, the physiological signal determination module 106 may determine that the user is out of breath or the action speed is too slow, and the alarm device 114 may deliver a warning signal, which may be vibration, sounds, etc. The display unit 116 may also display information indicating that the user is out of breath or the action speed is too slow. If a number of actions N_y is between 2-3, the user's breath and action are both steady. The control unit 110 or the alarm device 114 may not execute any functions, or the display unit 116 may display information indicating that the user's breath and action are steady enough.

The step of the physiological signal determination module 106 generating the measurement result according to the predefined data and the numbers of actions N_1-N_(n−1) is not only limited to the comparison of the numbers of actions N_1-N_(n−1) and the predefined data (e.g. comparing whether the number of actions between each two breaths is between 2-3). For example, the numbers of actions N_1-N_(n−1) measured according to the occurrence time of each physiological signal S_1-S_n may undergo further operations or computations, e.g. converting to an accumulated number of actions corresponding to the occurrence of multiple physiological signals (such as the number of actions between the first breath and the tenth breath), determining the trend of data variation (increasing or decreasing), estimating the number of occurrences of the physiological signal within an execution of the action, or calculating the average of the numbers of actions N_1-N_(n−1), etc. In an embodiment, the alarm device 114 may also deliver a warning signal when the physiological signal determination module 106 determines that two consecutive numbers of actions N_z, N_(z+1) have a large difference, in order to indicate that the user's breath or action is not steady. The alarm device 114 or the control unit 110 may also execute corresponding functions according to other computational results of the numbers of action times N_1-N_(n−1); this is not limited herein.

Please note that, in this embodiment, the status detection module 108 may include a G-sensor, pressure sensor, gyroscope or other related devices and technologies capable of detecting the user's action, in order to determine the number of actions N_1-N_(n−1) executed by the user between time points when the physiological signal is measured. The abovementioned number of actions may not only be the number of reps of a dumbbell raise, but may also correspond to a number of walking steps, running steps, rope skips or full revolutions when riding a bicycle.

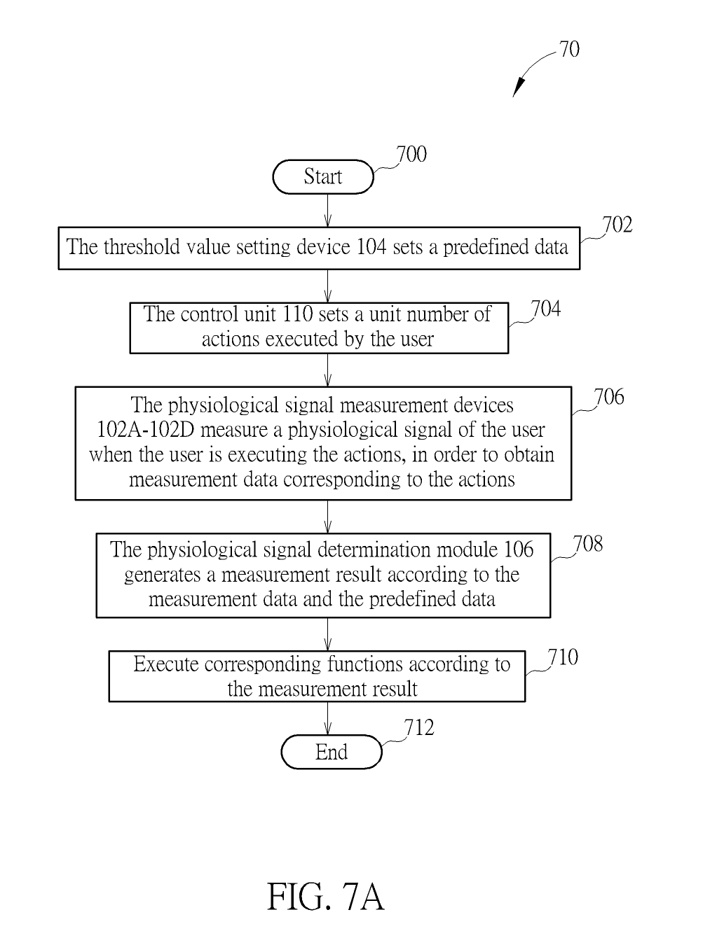

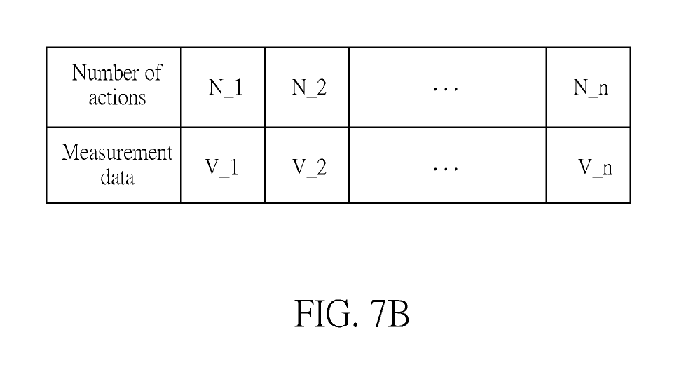

Please refer to FIG. 7A, which is a schematic diagram of a process 70 according to an embodiment of the present invention. As shown in FIG. 7A, the process 70 may be utilized in the wearable electronic device 10 for measuring the physiological signals of the user when the user is executing an action, in order to obtain the measurement data corresponding to a number of times the action is executed by the user. The process 70 includes the following steps:

Step 700: Start.

Step 702: The threshold value setting device 104 sets a predefined data.

Step 704: The control unit 110 sets a unit number of actions executed by the user.

Step 706: The physiological signal measurement devices 102A-102D measure a physiological signal of the user when the user is executing the actions, in order to obtain measurement data corresponding to the actions.

Step 708: The physiological signal determination module 106 generates a measurement result according to the measurement data and the predefined data.

Step 710: Execute corresponding functions according to the measurement result.

Step 712: End.

In the process 70, the threshold value setting device 104 first sets the predefined data corresponding to the physiological signal. The control unit 110 also predefines a unit number of actions executed by the user. For example, if the user needs to determine whether his/her heart rate is steady when cycling, the threshold value setting device 104 may predefine a criterion where the heart rate measured during a time interval when both feet tread 20 times should be between 15-30 beats. If the number of heart beats measured during the time interval when both feet tread 20 times is greater than 30, the user may be peddling too fast. If the number of heart beats measured during the time interval when both feet tread 20 times is smaller than 15, the user may not achieve health benefits of the exercise.

As shown in FIG. 7B, the physiological signal measurement devices 102A-102D measure the physiological signal of the user when the user executes unit numbers of actions N_1, N_2, . . . , N_n, in order to obtain measurement data V_1, V_2, . . . , V_n corresponding to each action N_1, N_2, . . . , N_n. For example, when the user starts to ride a bicycle, or after a period of time of riding, the heart rate sensor 102C may detect the number of heart beats during the time periods of the 1^{st} to 20^{th} treads, the 21^{st} to 40^{th} treads, the 41^{st} to 60^{th} treads, . . . , and the n^{th} to (n+19)^{th} treads, respectively. The physiological signal determination module 106 then generates the measurement result according to the measurement data V_1-V_n and the predefined data. The control unit 110, the alarm device 114 or the display unit 116 may further execute corresponding functions according to the measurement result. In the above embodiment, if the physiological signal determination module 106 determines that the number of heart beats measured during the time periods of performing 20 treads is greater than 30 or smaller than 15, the alarm device 114 may deliver a warning signal indicating that the user is peddling too fast or not fast enough, respectively, where the warning signal may be vibration, sounds, etc. The display unit 116 may also display information indicating that the user is peddling too fast or not fast enough. If the number of heart beats measured during the time period of performing 20 treads is between 15-30 beats, the physiological signal determination module 106 may determine that the user's exercise meets a requirement. The control unit 110 or the alarm device 114 may not execute any functions, or the display unit 116 may display information indicating that the user's exercise meets the requirement.

The step of the physiological signal determination module 106 generating the measurement result according to the measurement data V_1-V_n and the predefined data is not only limited to the comparison between the measurement data V_1-V_n and the predefined data (e.g. comparing whether the number of heart beats measured during the time period of executing the unit number of actions is between 15-30). For example, the measurement data V_1-V_n measured during the time period of executing the unit number of actions may undergo further operations or computations, e.g. determining the trend of data variation (increasing or decreasing), estimating measurement data corresponding to a unit number of actions executed in the future, or calculating the average of the data V_1-V_n, etc. The measurement data of different types of physiological signals obtained during the time period of executing each action may also be utilized for estimating other types of data. In an embodiment, the alarm device 114 may deliver a warning signal when the number of heart beats measured by the physiological signal determination module 106 during the execution time of a plurality of consecutive unit numbers of actions obviously increases, in order to indicate that the user's exercise rate is increasing too fast. The alarm device 114 or the control unit 110 may also execute corresponding functions according to other computational results of the measurement data V_1-V_n; this is not limited herein.

Similarly, in this embodiment, the status detection module 108 may include a G-sensor, pressure sensor, gyroscope or other related devices and technologies capable of detecting the user's action, in order to measure the number of actions executed by the user. This allows the control unit 110 to determine the physiological information of the user according to the actions executed by the user, which may further be utilized for determining the measurement data V_1-V_n of the user during the time period for executing each action N_1-N_n. The abovementioned unit number of actions may not only be the treading numbers of riding a bicycle, but may also be the number of walking steps, running steps, rope skipping times or dumbbell raise times, etc.

In the prior art, the wearable devices cannot simultaneously perform measurement on different types of physiological signals, and thus cannot combine the measurement data of different types of physiological signals or human actions. The information obtained by the conventional devices must first be transmitted to a processing means such as a computer or smart phone for data analysis and processing. Variations in the physiological information cannot be obtained immediately, which is inconvenient. In comparison, the method of measuring signals and the wearable electronic device of the present invention are capable of measuring various human physiological signals and a user's actions as well as generating related location information, to provide timely information for the user according to the measured data. In addition, when the user's physiological signals become abnormal, the user may carry out contingency plans in good time, which prevents the user from danger or physiological damage.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of measuring signals for a wearable electronic device comprising:
setting at least one threshold value corresponding to each of a plurality of physiological signals of a user;
measuring the plurality of physiological signals to obtain a plurality of measurement data; and
obtaining a measurement result according to the threshold value and the plurality of measurement data.

2. The method of claim 1, wherein the plurality of physiological signals comprise a breathing rate, a heart rate, a body temperature and brain waves of the user.

3. The method of claim 1, further comprising:
generating a warning signal according to the measurement result.

4. The method of claim 3, further comprising:
displaying the plurality of measurement data, the measurement result or the warning signal.

5. The method of claim 1, wherein the step of obtaining the measurement result according to the threshold value and the plurality of measurement data comprises:
determining a concentration of the user according to a breathing rate and brain waves of the user.

6. The method of claim 1, wherein the step of measuring the plurality of physiological signals to obtain the plurality of measurement data comprises:
measuring a physiological signal among the plurality of physiological signals of the user during a unit of time, in order to obtain a measurement data corresponding to the unit of time.

7. The method of claim 1, wherein the step of measuring the plurality of physiological signals to obtain the plurality of measurement data comprises:
measuring a physiological signal among the plurality of physiological signals of the user, in order to determine a time interval between an occurrence of the physiological signal and an occurrence of the next or later physiological signal.

8. A method of measuring signals for a wearable electronic device comprising:
setting at least one threshold value corresponding to each of at least one physiological signal of a user;
detecting a status of the user to generate a status signal;
measuring the physiological signal and obtaining at least one measurement data according to a relationship between the status signal and the physiological signal; and
obtaining a measurement result according to the threshold value, the status signal and the measurement data.

9. The method of claim 8, wherein the physiological signal comprises a breathing rate, a heart rate, a body temperature and brain waves of the user.

10. The method of claim 8, wherein the status comprises a location, an altitude or an action of the user.

11. The method of claim 8, further comprising:
generating a warning signal according to the measurement result.

12. The method of claim 11, further comprising:
displaying the plurality of measurement data, the status signal, the measurement result or the warning signal.

13. The method of claim 8, wherein the step of detecting the status of the user to generate the status signal comprises:
detecting a location of the user and determining a moving distance of the user according to the location of the user detected at different time points.

14. The method of claim 13, wherein the step of measuring the physiological signal and obtaining the measurement data according to the relationship between the status signal and the at least one physiological signal comprises:
setting a length of a unit of distance within the moving distance of the user; and
measuring a physiological signal among the at least one physiological signal of the user when the user is moving through the unit of distance within the moving distance, in order to obtain a measurement data corresponding to the unit of distance.

15. The method of claim 13, wherein the step of measuring the physiological signal and obtaining the measurement data according to the relationship between the status signal and the physiological signal comprises:
measuring a physiological signal among the physiological signal of the user, in order to determine the moving distance of the user during a time interval between an occurrence of the physiological signal and an occurrence of the previous or former physiological signal.

16. The method of claim 8, wherein the step of setting the threshold value corresponding to each of the physiological signal of the user comprises:
setting the threshold value for an altitude according to the altitude of the user.

17. The method of claim 8, wherein the step of obtaining the measurement result according to the threshold value, the status signal and the measurement data comprises:
obtaining measurement data corresponding to a first altitude and a second altitude when the user is located at the first altitude and the second altitude, respectively, and calculating a variation of the measurement data corresponding to an altitude variation between the first altitude and the second altitude.

18. The method of claim 8, wherein the step of detecting the status of the user to generate the status signal comprises:
detecting an action of the user, and determining a number of times the action is executed by the user according to an action status of the user at different time points.

19. The method of claim 18, wherein the step of measuring the at least one physiological signal and obtaining the measurement data according to the relationship between the status signal and the at least one physiological signal comprises:
measuring a physiological signal among the at least one physiological signal of the user, and determining the number of operations of the action executed by the user during a time interval between an occurrence of the physiological signal and an occurrence of the next or later physiological signal.

20. The method of claim 18, wherein the step of measuring the at least one physiological signal and obtaining the measurement data according to the relationship between the status signal and the at least one physiological signal comprises:
measuring a physiological signal among the at least one physiological signal of the user when the user is executing the action, in order to obtain a measurement data corresponding to the number of times the action is executed by the user.

21. A wearable electronic device, comprising:
a threshold value setting device, for setting at least one threshold value corresponding to each of at least one physiological signal;
at least one physiological signal measurement device, each for measuring each of the physiological signal, respectively, to obtain at least one measurement data; and
a physiological signal determination module, for obtaining a measurement result according to the threshold value and the measurement data.

22. The wearable electronic device of claim 21, further comprising:
at least one status detection module, for detecting a status of the user to generate a status signal.

23. The wearable electronic device of claim 22, wherein the status detection module comprises a global positioning system, a gravity sensor, a gyroscope and an atmospheric pressure detector.

24. The wearable electronic device of claim 21, further comprising:
an alarm device, for generating a warning signal according to the measurement result;
a display unit, for displaying the measurement data, the measurement result or the warning signal;
a control unit, for controlling operations of the wearable electronic device; and
a memory unit, for storing the measurement data and the measurement result.
